(19) United States
(12) Patent Application Publication
(10) Pub. No.: US 2017/0007480 A1
(43) Pub. Date: Jan. 12, 2017

(54) ERGONOMIC SYSTEM FOR AN ADJUSTABLE BED SYSTEM

(71) Applicant: LOGICDATA ELECTRONIC & SOFTWARE ENTWICKLUNGS GMBH, Deutschlandsberg (AT)

(72) Inventor: Walter KOCH, Schwanberg (AT)

(73) Assignee: LOGICDATA Electronic & Software Entwicklungs GmbH, Deutschlandsberg (AT)

(21) Appl. No.: 15/113,040

(22) PCT Filed: Jan. 16, 2015
(86) PCT No.: PCT/EP2015/050776

§ 371 Date: Jul. 20, 2016

(30) Foreign Application Priority Data
Jan. 20, 2014 (DE) 10 2014 100 548.1

(51) Int. Cl. A61G 7/018; G05B 15/02; A61B 5/00; A61B 5/11; A61G 7/015; A61G 7/057

(52) U.S. Cl. CPC A61G 7/018; A61G 7/015; A61G 7/057; A61B 5/0077; A61B 5/1113; A61B 5/1116; A61B 5/1128; A61B 5/4806; A61B 5/447; A61B 5/681; G05B 15/02; A61G 2203/10; A61G 2203/36; A61B 2560/0487; A61B 2562/0204

(57) ABSTRACT

The invention relates to an ergonomic system for an adjustable bed system, said ergonomic system having a data processing device and an image recording device coupled to the data processing device, said image recording device being arranged and configured in such a way that image data constituting an image of a user of the bed system are transmitted to the data processing device, wherein said data processing device is connected with an ergonomics database and is configured to evaluate the image data, in order to determine the user's position data and posture data. The data processing device is further configured to compare the determined position data and posture data with reference data in the ergonomics database, in order to determine a parameter for an ergonomic accuracy of a corresponding user position and posture according to the comparison. Furthermore, the data processing device is configured to adjust a bed system setting according to the determined parameter.

### BACKGROUND OF THE INVENTION

The disclosure relates to an ergonomic system for an adjustable bed system.

A healthy and recuperative sleep largely contributes towards preserving physical health. Modern adjustable bed systems, in which, for example, a headboard or footboard can be adjusted or in which sections of a mattress or of a mattress pad can be set to varying degrees of hardness or softness, are intended to contribute towards allowing a user such a healthy sleep. In the medicinal sector, in particular, such adjustable bed systems are necessary to assure a user an ideal sleeping posture or position after a surgical operation, for example, thus allowing said user to optimally regenerate.

In practice, however, users of such bed systems themselves rarely ever pay attention to an ergonomic sleeping posture or bed system setting. In many cases, users do not have the leisure to ideally adjust a bed system with respect to the ergonomics. On the other hand, for example, it is not possible for patients reliant upon so-called decubitus loungers to adjust their decubitus recliner due to the very fact of their physical limitations. A decubitus recliner is usually automatically adjusted in specific pre-determined patterns, but it is not always ideally adjusted ergonomically to the individual situation and the patient's preference.

### SUMMARY OF THE INVENTION

The present disclosure provides a flexible concept for an ergonomic system that can be used for an adjustable bed system.

The flexible concept for an ergonomic system is based on the idea of acquiring position data and posture date of a bed system user from image data, recorded via an image recording device, thus allowing a user's current ergonomic parameters to be recorded and evaluated. By comparing such data with reference data stored in an ergonomics database, for example, said data can be used to adjust a bed system setting, for example to adjust a headboard or footboard. Alternatively, sections of a reclining area can also be set to be harder or softer, by changing an air pressure in appropriate air cushions or air chambers of a mattress or a mattress pad, for example, thus allowing an automatic adjustment of the bed system.

The flexible concept also allows a user to actively change a bed system setting, by applying pre-determined gestures, which are recognizable by means of the data processing device, thus allowing a bed system setting to be adjusted according to the gestures.

In general, an ergonomic system is understood to be a system, which contributes towards detecting and influencing ergonomic parameters of a user or person, in order to prevent and/or treat health detriments or problems.

For example, the data processing device comprises a conventional workstation or a notebook. As an alternative, the data processing device can just as well comprise a mobile communication device, such as a smartphone or tablet, for example.

Another conceivable alternative is that the data processing device is designed as integrated unit with the image recording device. This can, for example, be used independently of the workstation. However, it is also possible to use a camera, which is integrated in the workstation or connected therewith, as image recording device. It is also possible to use a tablet with corresponding front or rear camera as data processing device with integrated image recording device.

In an exemplary embodiment, an ergonomic system for an adjustable bed system comprises a data processing device and an image recording device coupled to said data processing device. The image recording device is arranged and configured in such a way that image data constituting an image of a user of the bed system are transmitted to the data processing device. Said data processing device is connected with an ergonomics database and configured to evaluate image data, in order to determine the user's position data and posture data, to compare the determined position data and posture data with reference data in the ergonomics database, in order to determine a parameter for an ergonomic accuracy of an appropriate user position and posture according to the comparison. According to the determined parameter, the data processing device is further configured to adjust a bed system setting.

The ergonomic system provides that user position data and posture data are acquired from image data, said position data and posture data allowing a current posture, for example a sleeping posture or position, to be determined. Comparison with reference data in the ergonomics database allows the parameter as to what extent the user is in an ergonomically optimal or disadvantageous posture to be determined. Proceeding from this parameter, a bed system setting can now be adjusted, in order to improve the user's posture in an ergonomic respect. The ergonomic system thus allows the bed system setting to be automatically adjusted so that a user assumes an ergonomically more favourable position or posture. The adjustment of the setting can result in users being directly moved into a better position or that users are encouraged to move intuitively into such better position.

In one embodiment a reclining area of the bed system is set, in particular in sections, according to the determined hardness parameter, thus allowing a hardness of the reclining area to be adjusted according to a position and/or posture of a person on the reclining area, thus, for example, causing a person to assume an ergonomically more favourable posture and/or position or a person to lie more pleasantly in the current posture. In particular, for example, individual sections of the reclining area can be set to be of varying hardness. This can ensue, for example, by means of one or more air chambers of a mattress or a mattress pad, which can be filled with air or emptied to adjust the bed system setting, for instance the hardness of the mattress or mattress pad. This allows sections of a mattress or of a mattress pad to be set to be harder or softer, thus, for example, inducing a user to move into an ergonomically more favourable posture or position while sleeping.

Alternatively, the reclining area can also be set to varying degrees of hardness, at least in sections, by means of gel-like liquids, for example in a mattress or mattress pad. For example, gel granulate can be added to one or more closed chambers of a mattress or a mattress pad filled with liquid by means of a control device and appropriate actuators to make such sections harder. Conversely, salts can also be added to one or more such chambers, wherein the salts reverse the effect of the gel granulate, thus making the corresponding chambers softer again. In a further alternative, a plurality of tapes, such as cloth tapes, for example, can be provided for the reclining area, which can be tightened or relaxed by means of motors, so that specific sections of the reclining area become harder or softer.

According to a further embodiment, one or more adjusting motors of a bed frame of the bed system are actuated for adjusting the bed system setting according to the determined parameter, thus, for example, allowing a headboard and/or footboard of the bed system to be adjusted.

According to a further embodiment, the ergonomic system further comprises a motion sensor connected with the data processing device, said motion sensor being configured to be worn by a user, in particular integrated in a bracelet, and so that motion data constituting the motion of a user are transmitted to the data processing device. Said data processing device is configured to evaluate the motion data, in order to determine a user motion and adjust the bed system setting according to said motion, thus allowing a user's motion data, from which a user motion can be derived, to be recorded in addition to the image data. Combined with the user's position and posture data, this allows the parameter for the ergonomic accuracy to be particularly precisely determined. An alternative possibility is to simply adjust the bed system setting on the basis of the motion data of the motion sensor, without having to resort to the image data and/or determine the parameter for ergonomic accuracy.

According to a further embodiment, the image recording device and/or the motion sensor are wirelessly connected with the data processing device. This can, for example, be by means of a Bluetooth connection or a Wireless LAN connection.

The image recording device preferably comprises a digital camera, which in turn preferably has a video capture device to enable moving image data to be recorded. However, in principle, individual images recorded at specific intervals can also be used for the evaluation. Alternatively, it is also conceivable that the image recording device comprises a plurality of cameras, such as three cameras, for example, thus allowing a greater capture range, inter alia. In addition, it is possible to spatially detect or evaluate the user's position and posture, i.e. three-dimensionally, for example. Another alternative is to use infrared lighting instead of a camera for spatially detecting the user's position and posture. It should be pointed out that the image recording device is capable of recording the corresponding image data in the dark, i.e. at night, for example, which allows the data to be expediently evaluated. To this end, the image recording device itself can have an appropriate device, such as infrared lighting, for example.

According to a further embodiment, the data processing device is configured to implement gesture recognition by evaluating the image data and to adjust the bed system setting according to a recognized gesture, thus allowing a user to tell the ergonomic system by active participation, for example by active gesticulation, to change the bed system setting. It is possible, for example, to signalize that a headboard or footboard of the bed system is to be pivoted upwards or downwards by appropriate upwards and downwards movements of a hand. Gesture recognition thus allows the bed system to be manually rather than automatically adjusted. It should be noted that the parameter for ergonomic accuracy does not necessarily need to be determined according to the recognition of gestures. Also conceivable is the use of an ergonomic system, which comprises nothing but gesture recognition, as a basis for an adjustment of the bed system setting.

According to a further embodiment, the data processing device is configured to determine a user sleep phase according to the determined position and posture data and/or the determined movement. For example, it is possible to detect a deep sleep phase or a so-called REM (rapid eye movement) sleep phase, also designated as dream sleep phase.

The data processing device is preferably configured to adjust the bed system setting according to the determined sleep phase, in particular to wake a user. This allows the ergonomic system to automatically react to a determined user sleep phase and to adjust the bed system setting, thus also allowing a user to be woken within a specified timeframe in a particularly ergonomic, i.e. particularly pleasant, manner. For example, according to the determined sleep phase, the user can thus be woken by adjusting a headboard or a footboard or by alternate frequent change in the hardness or softness settings of at least a section of a reclining area. The user is thus galvanized into action, for example. Optionally, for example, various vibrations or vibration patterns can be generated for waking purposes in at least one section of the reclining area, for example by means of the initially mentioned (cloth) tapes or air cushions. Thus, for example, only the head areas of the reclining area can be changed for waking purposes. Another possible option is to use existing massage devices of the bed system for waking purposes.

### BRIEF DESCRIPTION OF THE DRAWINGS

In the following the invention is explained in detail on the basis of a plurality of embodiment with reference to the illustrations. In so doing, the embodiments are not restricted to their tangible representation. In addition, elements having the same effect or function bear the same reference sign, thus avoiding repeated explanations in individual cases.

Shown in:

FIG. 1 an embodiment of an ergonomic system with an adjustable bed system,

FIG. 2 a further embodiment of an ergonomic system and

FIG. 3 a further embodiment of an ergonomic system.

### DETAILED DESCRIPTION

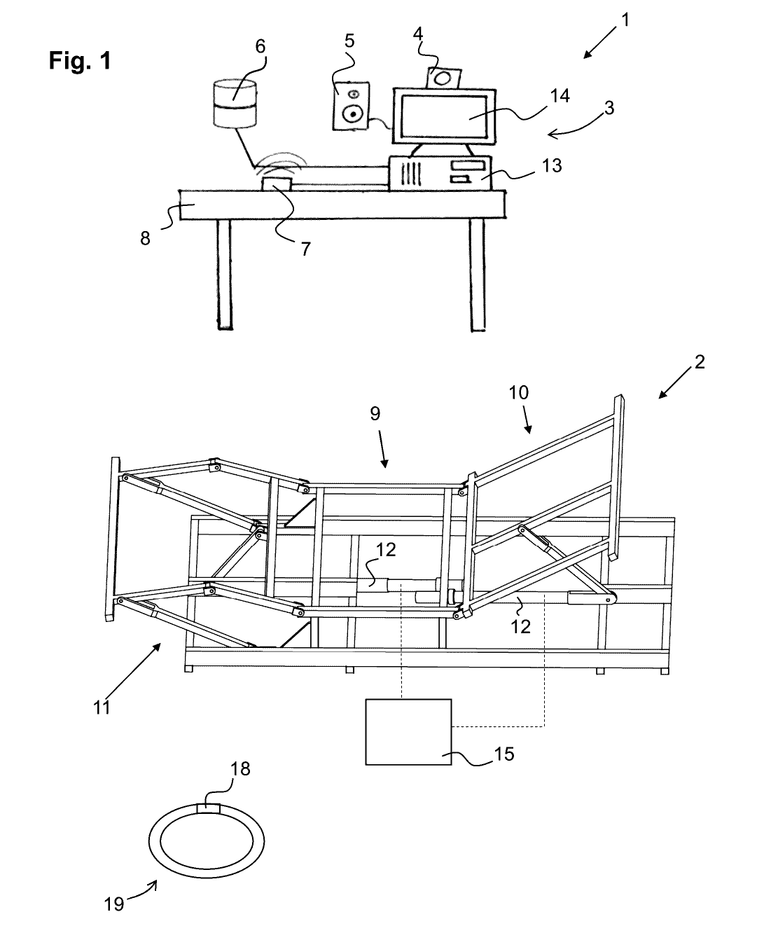

FIG. 1 shows an embodiment of an ergonomic system 1 with an adjustable bed system 2. A data processing device 3, an image recording device 4, audio means 5, a database 6 as well as an identification device 7 are shown. Such components can be arranged on a table 8, as shown in the embodiment. In addition to a stationary frame structure, the bed system 2 comprises an adjustable frame structure 9 with an adjustable headboard 10 and an adjustable footboard 11. The bed system 2 further comprises two motors 12 for adjusting the headboard 10 or footboard 11.

The data processing device 3 comprises a workstation 13 and a display device 14, the latter being designed as standard monitor of a PC workstation system, for example. Alternatively, the data processing device 3 can comprise only one workstation 13.

The ergonomic system 1 comprises the data processing device 3 and the image recording device 4, which are connected with each other. The database 6, used as ergonomics database, can be integrated in the ergonomic system 1 or arranged externally. Likewise, the identification device 7 and the audio means 5 can be either independent or assigned to the ergonomic system 1. The database 6 is preferably maintained centrally and accessible to the data processing device 3 via a network connection. The database 6 can also be designed as cloud database and arranged externally, thus allowing the data processing device 3 to access the database 6 via an existing Internet connection. Alternatively, the database 6 can also be provided by a mobile device, such as a smartphone or a tablet, wherein the data processing device 3 can be connected with the mobile end device via a network connection, preferably wireless.

For example, operating elements (not shown) can be provided in the bed system 2, which allow the motors 12 to be activated, in order to adjust the headboard 10 and/or the footboard 11 of the bed system 2. As an example, the headboard 10 can be set higher or lower. For example, it is also possible to move the frame structure 9 to a horizontal level with relation to the stationary frame structure by means of a motor 12 or of a further motor (not shown). This allows varied settings of the bed system 2 to be implemented, thus, for example, achieving ergonomically favourable conditions for a bed system 2 user located e.g. on a reclining area (not shown) of the bed system 2. The setting of ergonomically more favourable conditions is further supported by the ergonomic system 1.

The image recording device 4 is configured to transmit image data constituting a reflection of the bed system 2 user to the data processing device 3. In general, the image recording device 4 is advantageously oriented towards the bed system 2 user or to the bed system 2, in order to capture the entire bed system 2 or the user, where possible. The data processing device 3 is configured to evaluate the image data, in order to determine personal data, such as the user's position data and posture data. Said position data and posture data can, for example, specify whether users sleeping on a mattress of the bed systems 2 are lying on their back, side or in other positions on the mattress or in relation to the frame structure 9. Moreover, it can be determined where specific body parts, such as the shoulder or buttock, for example, are located in relation to each other or in relation to the frame structure 9. Appropriate software for determining the position data and posture data is, for example, implemented in the data processing device 3. Furthermore, the data processing device 3 is able to procure reference data from the database 6 and to compare such data with the determined personal data, in particular the user's position data and posture data. This comparison allows the data processing device 3 to specify a parameter for an ergonomic accuracy of an appropriate user position and posture. According to the determined parameter, the data processing device 3 is further configured to adjust or change a bed system 2 setting.

The adjustment of a bed system setting can, for example, comprise an adjustment of the headboard 10 or the footboard 11. To this end, the data processing device 3 can, for example, be coupled with a controller 15, which in turn is connected with the motors 12 (dashed line) and activates the motors 12. The coupling between the controller 15 and the data processing device can, for example, be wireless or cabled. The controller 15 is separately arranged outside the bed system 2 in FIG. 1. The controller 15 can, however, also be arranged on the frame structure 9 or integrated in the data processing device 3. An alternative possibility is that the controller 15 forms a drive unit together with a motor 12 in each case and is arranged in a joint housing with a motor 12.

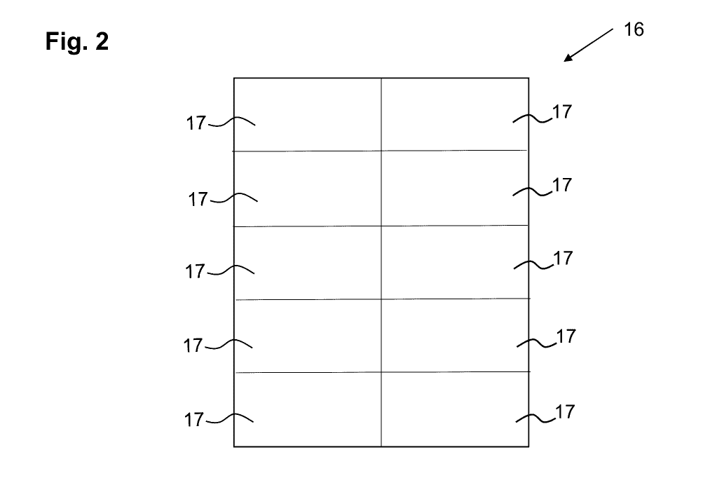

Further bed system 2 settings can apply to the reclining area (not shown) of the bed system 2, wherein, for example, different sections of the reclining area can be separately adjusted with respect to hardness (see also FIG. 2 with associated description). For example, components of the frame structure 9 or the initially mentioned (cloth) tapes, which can be tightened via motors or actuators, can serve to adjust the hardness of the reclining area. In addition, one or more sections of the reclining area can be lowered or raised by means of actuators such as the motors 12, thus allowing the buttock or shoulder of the person lying on the reclining area to be lowered. To this end, the controller 15 or a further controller can be provided, which achieves the lowering of the buttock or shoulder, by tightening or relaxing the (cloth) tapes, for example. Alternatively or additionally, the adjustment of the bed system 2 setting can also comprise the change in the hardness of the reclining area by means of gel granulate, as initially described.

FIG. 2 shows a further embodiment of an ergonomic system 1 with an adjustable bed system 2. The ergonomic system 1 essentially comprises the same features and functions as shown and described in conjunction with FIG. 1. For reasons of transparency, neither the ergonomic system 1 nor the bed system 2 are shown. FIG. 2 merely shows a schematic diagram of a mattress 16, which is, for example, mounted on the frame structure 9 and/or arranged on the latter. The mattress 16 can also constitute a mattress pad arranged on a mattress. Said mattress 16 constitutes a reclining area and comprises a plurality of air cushions 17. Said air cushions 17 each have an air chamber, which can be filled with air or emptied again via the controller 15 (see FIG. 1). In addition, the controller 15 can activate a compressor or an air pump, for example, which are connected with the individual air chambers.

It should be noted that the representation of the mattress 16 in FIG. 2 is merely schematic. The mattress 16 can comprise a much higher quantity of air cushions 17 and/or a different arrangement of the air cushions 17. For example, the air cushions 17 can be arranged parallel to each other in the longitudinal direction or in the transverse direction. Appropriate chambers or sections can also be used instead of the air cushions 17 when using gel granulate for setting the hardness of the reclining area. Cloth tapes can also be used to set the hardness, as described.

As already described on the basis of FIG. 1, the data processing device 3 can specify a parameter for an ergonomic accuracy of an appropriate position and posture of a user lying on the mattress 16. According to the determined parameter of the ergonomic accuracy, the air cushions 17 can now be varyingly filled with air or emptied, to enable varied air pressures to be applied to the air cushions 17. This means that the mattress 16 can have varyingly hard or soft sections or air cushions 17, thus, for example, possibly compelling the sleeping person to assume an ergonomic posture by changing the air pressures inside the air cushions 17, in that, for example, specific regions are set to be softer, so that part of the person's body sinks into a softer region and the overall body thus changes its posture and position. In so doing, the previously mentioned lowering of the buttock and/or shoulder can also be achieved. Furthermore, for example, it is possible to recognize when the sleeping person unintentionally moves away from the mattress 16. A corresponding change in the hardness in the peripheral area of the mattress 16, beyond which the person would like to move, can prevent the person falling off the mattress 16.

The ergonomic system 1 with such a mattress 16 can, for example, be used as a decubitus recliner, which detects when a person does not feel comfortable on a decubitus lounger or has assumed an ergonomically bad posture and would like to be moved into another posture or position. Due to the usually physical limitations of such persons, who are reliant upon decubitus loungers, it is not possible for the persons to change their posture independently. The described ergonomic system 1 allows the posture of such persons to be automatically adjusted.

The embodiments described on the basis of FIGS. 1 and 2 can also be combined in alternative embodiments, and therefore, for example, the bed system 2 comprises both the motors 12 for adjusting the headboard 10 or footboard 11 shown in FIG. 1 and the mattress 16 with the air cushions 17 shown in FIG. 2. In the process, the data processing device 3 is configured to both adjust the headboard 10 or footboard 11 and to varyingly fill the air cushions 17 with air or empty them, according to the parameter for an ergonomic accuracy.

In an alternative embodiment, the ergonomic system 1 can comprise a motion sensor 18 connected with the data processing device 3, which is integrated in a bracelet 19 (see FIG. 1). The bracelet 19 can be worn by a user and is configured to transmit motion data constituting a motion of a user to the data processing device 3. Said data processing device 3 is configured to evaluate the motion data, in order to determine a user motion and to adjust a bed system 2 setting according to the motion and according to the functionalities presented in FIGS. 1 and 2.

The motion sensor 18 can thus be used in combination with the image recording device 4, thus allowing the parameter for an ergonomic accuracy of an appropriate user position and posture to be particularly precisely specified. For example, whether a user moves away from the bed system 2 or the mattress 16 can be detected. According to such a movement, the data processing device 3 can now adjust the air cushions 17 of the mattress 16 and/or the headboard 10 or footboard 11 via the controller 15, in order, for example, to prevent the person falling onto the floor from the mattress 16 or from the frame structure 9.

As described, the motion sensor 18 is used in addition to the image recording device 4 in the ergonomic system 1. Said motion sensor 18 can, however, also be used separately to the image recording device 4 in a system having no image recording device 4. In such system the data processing device 3 is configured in such a way as to adjust a bed system 2 setting merely according to the motion sensor 18. The data processing device 3 does not necessarily need to determine a parameter for the ergonomic accuracy either, and can thus decide simply on the basis of the evaluated motion data whether a bed system 2 setting needs to be made.

Optionally, the ergonomic system 1 according to FIGS. 1 and 2 is also configured to implement a user validation. This can be implemented by the data processing device 3 on the basis of the image data. Alternatively, a user validation can also ensue via an access medium, which is, for example, implemented via the identification device 7. For example, the user has a data card or device, which is configured for near field communication (NFC). In principle, however, a registration in the ergonomic system 1 with user name and password is also possible via the workstation 13.

If the ergonomic system 1 accordingly recognizes a user, personalized ergonomics data can be stored and retrieved on the basis of the user validation. This can, for instance, ensue in the database 6, but also in a separate storage system as an alternative. The parameter for the accuracy of the user's position and posture is preferably additionally specified on the basis of the personalized ergonomics data. For example, said personalized ergonomics data can comprise profiles of motion data, which have been recorded by means of the motion sensors in past sleep periods. Thus, for example, the data processing device 3 allows a pattern corresponding to the user's normal sleeping habits to be detected, thus in turn allowing probably occurring user movements to be countered during sleep in advance on the basis of time.

In principle, in addition to a user validation, the ergonomic system 1 is also able to recognize a user's absence from the bed system 2. For example, the ergonomic system 1 can detect the absence of a user and, if necessary, take energy-saving measures, for example the discontinuation of various services or of the database 6. It is also conceivable that the ergonomic system 1 is coupled with external devices or components. For example, the ergonomic system 1 can be connected to house systems, such as a radiator or venetian blinds, and actuate the latter accordingly. Thus, for example, once a person's absence is recognized, the radiator can be switched off accordingly to save energy.

The ergonomic systems 1 described on the basis of FIG. 1 und 2 are substantially arranged on the table 8. Alternatively, various arrangements of the ergonomic system 1 are conceivable. For example, the ergonomic system 1 can be arranged on a nightstand or on a shelf or mounted on the wall. It is also conceivable that such an ergonomic system 1 is fitted to the stationary frame structure or to the adjustable frame structure 9 of the bed system 2.

In a further embodiment, which essentially comprises the features and functions described on the basis of FIG. 1 und 2, the data processing device 3 can be configured to implement gesture recognition by evaluating the image data and/or the motion data of the motion sensor 18 and to adjust a bed system 2 setting according to a recognized gesture and according to the description on the basis of FIGS. 1 and 2. Such an ergonomic system 1 can, for example, also implement the gesture recognition in addition to the determination of a parameter for an ergonomic accuracy. For example, the gesture recognition allows a user to adjust the height of the headboard 10 or the footboard 11 in the bed system 2 according to FIG. 1 by means of gesticulation. Alternatively or additionally, it is also possible for a user to change the pressure in specific air cushions 17 or to adjust other described settings by gesticulating as well. It could thus be that a bed system 2 setting is not made automatically, but instead by active participation on a user's part. It should be pointed out that the gesture recognition can also ensue solely on the basis of the motion data of the motion sensor 18. An ergonomic system 1 can also be designed only for such gesture recognition, wherein bed system 2 settings are not automatically adjusted by evaluating the image data.

The data processing device 3 described on the basis of FIGS. 1 and 2 can also be configured to determine a user sleep phase according to the determined position and posture data and/or the determined movement. By evaluating such data it is thus, for example, possible to recognize whether a user is in a deep sleep phase or in a dream phase. The determination of the sleep phase can, for example, be implemented for statistical purposes, wherein such statistics can be displayed via the display device 14, for example.

Optionally, the data processing device 3 can be further configured to adjust a bed system setting according to the determined sleep phases, in particular to wake a user. For example, it is thus possible to ergonomically optimally wake a user during a time period previously specified by the user, i.e. in an appropriate sleep phase. Such a previously specified time period when the user would like to be wakened can, for example, be from 6.30 to 7.00 am in the morning. If the user is in a light sleep phase during this time period, such as the dream sleep phase, for example, this can be detected by means of the data processing device 3 and the user wakened, for example, by an abrupt adjustment of the headboard 10 or the footboard 11, by frequent and rapid filling and emptying of the air cushions or by means of the described actuation of cloth tapes.

Vibrations or vibration patterns can be generated by the cited measures. The user can thus, for example, be already wakened at 6.35 am. If the ergonomic system 1 recognizes that the user is in a deep sleep phase, it is obliged not to wake the user at the moment but to do so by no later than 7 o'clock.

Optionally, a further embodiment of the ergonomic system 1 can comprise a microphone, which is arranged and configured in such a way that audio data representing sound signals of a user are transmitted to the data processing device 4 and said data processing device 3 is further configured to evaluate the audio data, in order to determine an audio pattern and to adjust a bed system 2 setting according to the audio muster. For example, a snore can thus be recognized as an audio pattern. If a snore is recognized, the bed system 2 can be adjusted according to the preceding descriptions. For example, a change in the user's position on the bed system 2 can be enforced, so that the snoring stops. This can also be achieved by generating vibrations as described above.

In a further embodiment, the data processing device 3 can be connected with audio means 5, as shown in FIG. 1, and configured to generate audio signals according to the determined position and posture data, the determined sleep phases and/or the determined audio patterns and output said audio signals via the audio means 5. For instance, a wake-up tone or wake-up melody can thus be signalized to wake a user.

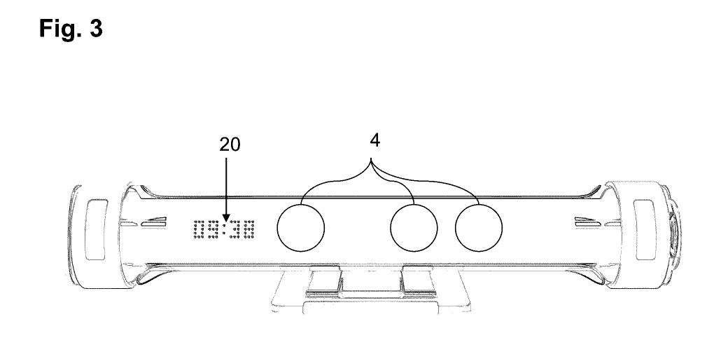

FIG. 3 shows an exemplary implementation of an ergonomic system 1, in which the data processing device 3 and the image recording device 4 are integrated in a joint housing. The housing is also provided with a display device 20. For example, a time of day can be output by means of the display device 20, as shown in FIG. 3.

In the embodiment shown, the image recording device 4 comprises a plurality of, in particular three, cameras, which each supply image data from which spatial characteristics can also be better derived. The intention is also to use two cameras for the spatial image detection and to arrange infrared lighting in the third place or circular hole for a better spatial detection by means of the reflected light.

For instance, the ergonomic system in the form shown can be connected with a work-station, such as the workstation 13 according to FIG. 1, for example, via a cabled connection or a wireless connection. A connection to a tablet or to another electronic computer can also be established. For example, an appropriate application is also installed on the tablet computer.

The ergonomic system 1 shown in FIG. 3 can be used in many different ways for a bed system 2, wherein the latter can be positioned in a particularly space-saving manner in the region of the bed system 2. Such an ergonomic system 1 can, for example, be secured to the frame structure 9 or on a wall of a room.

The ergonomic systems described on the basis of the embodiments uses varying data, such as image data, positions and/or posture data, audio samples or the sleep phases, for example. Such data can, for example, be statistically evaluated by means of the data processing device 3 and visualized, if necessary. Thus, for example, sleep statistics can be created and/or other evaluations made, such as the determination of snoring frequency.

It should also be pointed out that the used data, in particular the image data, does not necessarily need to be stored for reasons of data protection, and therefore no interventions in users' privacy rights is possible. For example, the data can be used for the evaluation and immediately deleted after evaluation.

The various previously described embodiments of the ergonomic system 1 are meant to encourage a user to permanent ergonomic behaviour. The use of the image recording device 4 and of the image data with corresponding evaluation by the data processing device 3 thus closes an ergonomic control loop with the user so to speak.

1. An ergonomic system for an adjustable bed system, said ergonomic system having a data processing device and an image recording device coupled to the data processing device, said image recording device being arranged and configured in such a way that image data constituting an image of a user of the bed system are transmitted to the data processing device, wherein said data processing device is connected with an ergonomics database and is configured
to evaluate the image data, in order to determine the user's position data and posture data;
to compare the determined position data and posture data with reference data in the ergonomics database, in order to determine a parameter for an ergonomic accuracy of an appropriate user position and posture according to the comparison; and
to adjust a bed systems setting according to the determined parameter.

2. The ergonomic system according to claim 1, wherein a hardness of a reclining area of the bed system, is adjusted, in particular in sections, according to the determined parameter for adjusting the bed system setting.

3. The ergonomic system according to claim 1,
wherein one or more adjusting motors of a bed frame of the bed system are actuated for adjusting the setting of the bed system according to the determined parameter.

4. The ergonomic system according to claim 1,
further comprising a motion sensor coupled to the data processing device, said motion sensor being configured to be worn by a user, in particular integrated in a bracelet, and so that motion data reflecting a user motion are transferred to the data processing device, wherein said data processing device is configured to evaluate the motion data, in order to determine a user motion and to adjust the bed system setting according to the motion.

5. The ergonomic system according to claim 1,
wherein the image recording device is wirelessly connected with the data processing device.

6. The ergonomic system according to claim 1,
wherein the image recording device comprises a digital camera, in particular with a video capture function.

7. The ergonomic system according to claim 1,
wherein the data processing device is configured to implement a user validation according to the image data and to store and retrieve personalized ergonomics data according to the user validation.

8. The ergonomic system according to claim 1,
wherein the data processing device is configured to implement a user validation via an access medium or a near-field communication, as well as to store and retrieve ergonomics data according to the user validation.

9. The ergonomic system according to claim 7, wherein the data processing device is configured to specify the parameter on the additional basis of the personalised ergonomics data, in particular the user's position, posture and weight.

10. The ergonomic system according to claim 1,
wherein the data processing device is configured to implement gesture recognition by evaluating the image data and to adjust the bed system setting according to a recognized gesture.

11. The ergonomic system according to claim 1,
wherein the data processing system is configured to determine a phase of sleep according to the determined position and posture data and/or the determined motion.

12. The ergonomic system according to claim 11, wherein the data processing device is further configured to adjust the bed system setting according to the determined phase of sleep, in particular to wake up a user.

13. The ergonomic system according to claim 1,
further comprising a microphone which is configured in such a way that audio data, representing sound signals of a user, are transmitted to the data processing device, and the data processing device is further configured to evaluate the audio data, in order to determine an audio pattern and to adjust the bed systems setting according to the audio pattern.

14. The ergonomic system according to claim 1,
wherein the data processing device is connected with audio means and is configured to generate sound signals according to at least one of the following types of data and to output said audio signals via the audio means:
the determined position and posture data;
the determined sleep phase; and
the determined audio pattern.

15. The ergonomic system according to claim 1,
wherein the data processing device is configured to detect any user absence.

16. The ergonomic system according to claim 4,
wherein the motion sensor is wirelessly connected with the data processing device.
